(19) United States
(12) Patent Application Publication
(10) Pub. No.: US 2023/0338083 A1
(43) Pub. Date: Oct. 26, 2023

(54) Catheter device for performing a cardiac sympathetic denervation intervention

(71) Applicant: BIOTRONIK AG, Buelach (CH)

(72) Inventors: Georg Nollert, Strasslach (DE); Tobias Kissling, Bern (CH)

(21) Appl. No.: 18/245,805

(22) PCT Filed: Oct. 4, 2021
(86) PCT No.: PCT/EP2021/077281

§ 371 Date: Mar. 17, 2023

(30) Foreign Application Priority Data
Oct. 8, 2020 (EP) 20200662.3

(51) Int. Cl. A61B 18/14; A61B 18/06; A61F 2/01

(52) U.S. Cl. CPC A61B 18/1492; A61B 18/06; A61F 2/014; A61B 2018/00214

(57) ABSTRACT

A catheter device for performing a cardiac sympathetic denervation intervention includes an ablation device for applying an ablation action. The ablation device has an expansion member that is expandable from a compressed delivery state to an expanded ablation state. The expansion member includes an inner lumen defining a blood flow passage through the ablation device in the expanded ablation state of the expansion member in an antegrade flow direction (F). The catheter device is configured for introduction into the aorta to perform a cardiac sympathetic denervation intervention at an ablation site within the aorta. The expansion member is configured to abut, in the expanded ablation state, an aortic wall section (W) and carries an ablation arrangement for applying ablation action at the ablation site.

### PRIORITY CLAIM

This application is a 35 U.S.C. 371 US National Phase and claims priority under 35 U.S.C. § 119, 35 U.S.C. 365(b) and all applicable statutes and treaties from prior PCT Application PCT/EP2021/077281, which was filed Oct. 4, 2021, which application claimed priority from European Application Serial Number 20200662.3, which was filed Oct. 8, 2020.

### FIELD OF THE INVENTION

The invention concerns cardiac sympathetic denervation devices.

### BACKGROUND

Cardiac sympathetic denervation serves to treat diseases which at least are influenced by sympathetic innervation. Such diseases may include heart failure, coronary artery disease, arrhythmias, stress-induced diseases, and hypertension. Further potentially treatable diseases include kidney insufficiency, diabetes mellitus, metabolic syndrome, sleeping disorders, and a treatment for the prevention of strokes.

Preganglionic sympathetic neurons extend from the lateral horn of the thoracic spinal cord to ganglia of the sympathetic chain, in particular to the ganglion stellatum and cervical ganglia. The sympathetic neurons form three cardiac nerves (superior, middle, inferior) and Rami cardiaci thoracici, which extend to the cardiac plexus, from which the nerves run alongside the great arteries (ascending aorta and pulmonary trunk) to the base of the heart and accompany the coronaries. Originally, a cardiac sympathetic denervation procedure has been carried out within a surgical procedure, commonly done in a minimally invasive thoracoscopic procedure. During the denervation procedure the lower half of the ganglion stellatum together with two caudal sympathetic ganglia on the left side or bilaterally are typically resected, or alternatively, as a temporary intervention the ganglion stellatum is blocked by injecting locally acting anesthetics.

There is a desire to perform a sympathetic denervation in a minimally invasive way using a catheter instead of a surgical operation. For this, a catheter device including an ablation device may be used to perform an ablative procedure inducing a local denaturation of tissue at an ablation site. The ablation site herein, for the sympathetic denervation, shall be the adventitia of the ascending aorta and pulmonary trunk and the space between the two vessels in order to interrupt the sympathetic nerves on their paths along the coronaries from the base of the heart.

US 2018/0161577 A1 discloses a device including an expandable structure including a plurality of splines extending from a proximal hub to a distal hub, a first electrode on a first spline of the plurality of splines, an outer tube extending from a handle to the proximal hub, and a shaft extending through the outer tube from the handle to the distal hub. The device is inserted in the pulmonary artery and operative for treatments of acute heart failure by neuromodulation using electric stimuli to sympathetic cardiac nerves.

US 2018/0147408 A1 and US 2009/0171411 A1 each disclose methods and systems of treating acute heart failure by applying a therapy signal to at least one sympathetic cardiopulmonary fiber surrounding the primary trunk that affects heart contractility more than heart rate. An electrode assembly herein includes a frame which is collapsible for fitting within a catheter lumen during insertion into the patient's body. The frame, when deployed, assumes a radially expanded configuration designed to contact a vessel wall at a site in the pulmonary artery.

These devices, as other devices primarily used in the pulmonary artery are introduced in an antegrade direction through the heart into the pulmonary artery, in particular the pulmonary trunk. Using ablation devices in the pulmonary artery, sympathetic nerves running along the pulmonary artery can be affected, which mainly modulate the sympathetic tone of the pulmonary arteries (inducing vasoconstriction and pulmonary hypertonus). However, ablation procedures in the pulmonary artery may not provide sufficient sympathetic denervation such that different procedures may have to be employed.

### SUMMARY OF THE INVENTION

A preferred catheter device can perform a cardiac sympathetic denervation intervention and includes an ablation device for applying an ablation action. The ablation device has an expansion member that is expandable from a compressed delivery state to an expanded ablation state. The expansion member includes an inner lumen defining a blood flow passage through the ablation device in the expanded ablation state of the expansion member in an antegrade flow direction (F). The catheter device is configured for introduction into the aorta to perform a cardiac sympathetic denervation intervention at an ablation site within the aorta. The expansion member is configured to abut, in the expanded ablation state, an aortic wall section (W) and carries an ablation arrangement for applying ablation action at the ablation site. The catheter device allows for an effective denervation in particular in the vicinity of the cardiac plexus located around the ascending aorta and in the space between the ascending aorta and pulmonary trunk.

### BRIEF DESCRIPTION OF THE DRAWINGS

The invention shall subsequently be described in more detail with reference to the embodiments shown in the figures. Herein:

FIG. 1 shows a view of a patient's heart, indicating the cardiac plexus located between the ascending aorta and the pulmonary trunk;

FIG. 2 shows a posterior view of a patient's heart visualizing the close anatomical relationship of the pulmonary artery and the ascending aorta. The cardiac sympathetic nerves travel within the adventitia of the great arteries and in the space in between, which is not separated by the pericardium;

FIG. 3 shows a coronal plane of the ascending aorta and the native aortic valve;

FIG. 4 shows a view of a patient's heart with an ablation device arranged in the aorta to perform an ablation;

FIG. 5 shows a view of an embodiment of an ablation device of a catheter device;

FIG. 6 shows a front view of the ablation device;

FIG. 7 shows a schematic drawing of another embodiment of a catheter device;

FIG. 8 shows a schematic view of yet another embodiment of a catheter device;

FIG. 9 shows a schematic drawing of yet another embodiment of a catheter device;

FIG. 10 shows a schematic view of yet another embodiment of a catheter device;

FIG. 11A-C show views of a rotational orientation indicator for indicating a rotational orientation of a catheter device;

FIG. 12 shows a schematic view of an embodiment of an ablation device;

FIG. 13 shows a schematic view of another embodiment of an ablation device;

FIG. 14 shows a schematic drawing of another embodiment of an ablation device;

FIG. 15 shows a schematic view of another embodiment of an ablation device;

FIG. 16 shows a schematic drawing of a catheter device as a whole; and

FIG. 17 shows a schematic view of a system including two catheter devices for an ablation both within the aorta and within the pulmonary artery of a patient's heart.

### DETAILED DESCRIPTION OF THE PREFERRED EMBODIMENTS

A catheter device is provided which includes an ablation device for applying an ablation action, the ablation device including an expansion member which is expandable from a compressed delivery state to an expanded ablation state and which includes an inner lumen providing for a blood flow passage through the ablation device in the expanded ablation state of the expansion member in an antegrade flow direction. The catheter device is configured for introduction into the aorta to perform a cardiac sympathetic denervation intervention at an ablation site within the aorta, wherein the expansion member is configured to abut, in the expanded ablation state, an aortic wall section and carries an ablation arrangement for applying said ablation action at the ablation site.

The catheter device hence is configured to be introduced into the aorta to achieve a cardiac sympathetic denervation. The catheter device, with its ablation device, in particular may be introduced into the aorta ascendens, which may be achieved by introducing the ablation device in a retrograde direction opposite to the antegrade blood flow into the aorta towards the ablation site. Ablation in this way may be performed within the aorta in a region distal to the aortic valve such that sympathetic nerves running in the adventitia of the ascending aorta or in the space between the ascending aorta and the pulmonary trunk may be affected for achieving a sympathetic denervation.

The normal aorta of an adult may have a diameter in between 2.5 cm to 4.5 cm. The catheter device, with its ablation device, is configured to be introduced in a retrograde direction towards the ablation site within the aorta, wherein for the delivery the expansion member of the ablation device assumes its compressed delivery state such that the ablation device may be easily moved towards the ablation site. At the ablation site, the expansion member is expanded to assume the expanded ablation state, in which the expansion member abuts the aortic wall at the ablation site such that the ablation arrangement carried on the expansion member comes into contact with the aortic wall and may carry out an ablation procedure at the ablation site.

The expansion member herein defines a blood flow passage on its inside, such that blood may flow through the expansion member in the expanded ablation state. The aorta hence is not blocked during the ablation procedure. Thus, even during the ablation blood may continue to flow through the aorta.

In the expanded ablation state the expansion member is in abutment with the aortic wall such that the expansion member and with it the ablation arrangement arranged on the expansion member is seated within the aorta to withstand the blood pressure within the aorta. Hence, using the catheter device an ablation within the aorta may be performed, which may cause a sympathetic denervation in the vicinity of the cardiac plexus alone or in combination with an additional ablation within the pulmonary artery.

In one embodiment, the catheter device includes a first catheter shaft and a second catheter shaft received within the first catheter shaft and being axially movable within the first catheter shaft. The ablation device is fixed to the second catheter shaft such that the ablation device is axially movable with respect to the first catheter shaft. By moving the second catheter shaft with the ablation device arranged thereon with respect to the first catheter device the expansion member may be brought into a position in which it may expand from its compressed delivery state to the expanded ablation state. Furthermore, by axially moving the second catheter shaft with the ablation device arranged thereon the axial position of the ablation device prior to the expansion of the expansion member may be adjusted in order to place the ablation device at the ablation site in a precise manner for performing an ablative procedure.

In one embodiment, the first catheter shaft includes a receptacle for receiving the expansion member in the compressed delivery state, wherein the expansion member, by axially moving the second catheter shaft relative to the first catheter shaft, is deliverable from the receptacle to transfer the expansion member from the compressed delivery state to the expanded ablation state. The receptacle may for example have the shape of a cylindrical housing in which the expansion member is received in the compressed delivery state. The receptacle hence provides for a sheathing of the expansion member, the sheathing being removable by axially displacing the second catheter shaft with respect to the first catheter shaft in order to move the ablation device out of the receptacle. Preferably the receptacle is flexible along its longitudinal direction to support the movement of the ablation device through narrow curves of a vessel.

The receptacle herein also allows for a re-sheathing after completing the ablative procedure, such that the ablation device may again be removed from the patient.

In one embodiment, the expansion member is self-expandable. For example, the expansion member may be made from an elastic material, such as a nitinol material, and may be formed from a stent-like structure like a frame cut from a tube or a wireframe made from wires, having for example a cell structure in which closed cells are formed by an arrangement of struts. Once the expansion member is moved out of the receptacle of the first catheter shaft and hence no longer is maintained in its compressed delivery state, the expansion member self-expands and hence transitions from the compressed delivery state to the expanded ablation state, without an additional, external force being required to cause the expansion of the expansion member.

In an alternative embodiment the expansion member is inflatable using a fluid to expand from the compressed delivery state to the expanded ablation state. The expansion member, according to this embodiment, hence is not self-expandable, but is caused to expand by inflating the expansion member. For this, the expansion member for example may have the shape of an inflatable balloon, which however is hollow at its inside and hence defines an inner lumen for providing for a blood flow passage in the expanded state of the expansion member.

The expansion member may have a substantially cylindrical shape, the expansion member hence extending along the antegrade flow direction along a length which may for example be larger than a diameter of the expansion member in the expanded state (with respect to a non-deformed state, a deformation for example being caused by the interaction with the aortic wall during placement of the expansion member within the aorta ascendens at the ablation site).

In another embodiment, the expansion member may have the shape of a torus extending about the antegrade flow direction.

If the expansion member is inflatable by delivering a fluid to the expansion member, for example a fluid line is connected to the expansion member, the fluid line being formed for example on the second catheter shaft such that the fluid line runs along the second catheter shaft. If the expansion member shall be expanded, a fluid, such as a gas or a liquid fluid, is delivered to the expansion member and the expansion member hence is inflated to transition from the compressed delivery state to the expanded ablation state.

An inflation pressure of the expansion member herein may be controlled in order to control an abutment force by which the expansion member abuts the aortic wall in the expanded ablation state.

In one embodiment, the ablation arrangement is arranged on an outer face of the expansion member such that, when the expansion member is in the expanded ablation state, the ablation arrangement abuts the aortic wall for providing for an ablation on the aortic wall. The ablation arrangement may for example extend circumferentially about the expansion member and hence circumferentially about the antegrade flow direction, such that an ablation action may be exerted around the circumference of the expansion member.

The ablation arrangement may for example have the shape of a closed ring extending about the expansion member, the ablation arrangement serving to for example perform a cryoablation or a radiofrequency ablation along the circumference of the expansion member. By forming the ablation arrangement to have a rotationally symmetric shape the ablation device is insensitive with respect to a rotational orientation of the catheter device, making the placement of the ablation device at the ablation site easy and comfortable.

The ablation device may be configured to perform a cryoablation, a radiofrequency ablation, a microwave ablation, an ultrasound ablation, and/or a chemical ablation.

Within a cryoablation tissue is locally cooled, for example to temperatures below −20° C., for example below −40° C., more particularly to temperatures below −50° C. Cryoablation may for example be performed by an ablation arrangement through which a cooling agent, such as liquid carbon dioxide, may be delivered. In this case a fluid line is connected to the ablation arrangement for delivering the cooling agent towards the ablation arrangement, the fluid line being arranged on the second catheter shaft and running along the second catheter shaft for delivering the cooling agent towards the ablation arrangement.

Alternatively, a cryoablation may be performed using Peltier elements providing for an electro-thermal transformation when applying a voltage. In this case, electrical lines are connected to the ablation arrangement to control operation of the Peltier elements.

Within a radiofrequency ablation, the ablation arrangement is formed by one or multiple electrodes configured to inject a radiofrequency signal into the tissue in order to cause a local heating and hence an ablation of tissue to achieve a sympathetic denervation.

Radiofrequency ablation provides for a thermal ablation by ablating tissue using a local heating. Another thermal ablation procedure is microwave ablation by injecting a microwave signal into tissue.

Yet another thermal ablation technique which may be employed is ultrasound ablation, in particular high-intensity focused ultrasound ablation (in short HIFU), causing a local heating of tissue by injecting ultrasound energy.

Alternatively or in addition, chemical ablation may be used in which a chemical agent, for example alcohol, e.g. ethanol, is injected into tissue. For this, the ablation arrangement may be configured for an injection of the chemical agent into tissue and for this may include delivery openings, for example in the shape of micro-needles, allowing to inject the chemical agent into the tissue. Needle-shaped injection devices may for example have a length allowing to access the sympathetic nerves, such that by penetration of the aortic wall the sympathetic nerves may be reached and may be directly affected by injection of the chemical agent. Delivery of the chemical agent may for example be provided using microfluidics techniques, such as micro-channels, micro-pumps, micro-valves and the like, in order to control a flow of the chemical agent.

In one embodiment, the ablation arrangement includes a multiplicity of ablation members configured to abut with the aortic wall section at the ablation site. Multiple ablation members, for example in the shape of cooling pads to be cooled by a cooling fluid or cooling elements such as Peltier elements, or in the shape of electrodes for injecting a radiofrequency or microwave signal, or in the shape of chemical delivery devices such as needles, may be distributed over the external surface of the expansion member to form an arrangement at the outside of the expansion member for abutting with the aortic wall in the expanded state of the expansion member.

Using such arrangement of ablation members, a control of ablation becomes possible by controlling operation of the ablation members individually. For this, the catheter device for example may include a control device which is configured to independently control the different ablation members, for example by performing a cryoablation using the separate ablation members or by injecting radiofrequency signals using the different ablation members. In this way it may be achieved that an ablation may be caused on defined portions of the aortic wall, hence making it possible to locally confine an ablation action in a controlled manner. Hence, regions which shall not be subject to ablation may not be affected and hence may be preserved.

An individual control of the ablation members may make it necessary to place the ablation device at the ablation site in a defined rotational orientation. For this, the catheter device may for example include a rotational orientation indicator, for example placed at a catheter tip, allowing to monitor the rotational orientation of the catheter within the patient for example using an X-ray technique or the like. The rotational orientation indicator may for example include different indicator sections formed for example from a radiopaque material, which indicate a correct rotational orientation if they are brought, using a suitable imaging technique such as an X-ray technique, into a defined alignment with respect to one another.

Using an independent control of the ablation members of the ablation arrangement also a closed-loop operation becomes possible, in which a control of the individual ablation members can be controlled automatically in particular by independently controlling an ablation intensity caused by the individual ablation members. Such control may be supported for example by a mapping device, which allows to perform a locally defined and confined ablation action for the sympathetic denervation and to also monitor a progress of the ablation at the sympathetic nerves. The expansion member may include mapping electrodes to form a mapping device. The mapping electrodes allow a locally defined monitoring of the electric activity of the tissue in the region around the expansion member.

In one embodiment, the expansion member includes a filter element for filtering a blood flow passing through the inner lumen to remove particles from the blood flow. Such filter element may span for example across the inner lumen of the expansion member, such that particles within the blood flow, for example emboli, may be trapped and prevented from being transported within the blood flow.

Alternatively or in addition, the expansion member includes a valve arrangement including one or multiple leaflets for providing an artificial valve within the inner lumen of the expansion member. Such valve arrangement may in particular be suitable if the expansion member, for performing the ablation action, is placed within the native aortic valve, such that a proper functioning of the aortic valve during an ablation procedure cannot be ensured. In this case the aortic valve functionality is provided by the valve arrangement of the ablation device.

Alternatively or in addition, the expansion member includes an electrode arrangement configured to conduct impedance measurements in the vicinity of the ablation site. For example, a first electrode may be placed at a distal position with respect to the ablation arrangement, whereas a second electrode is placed at a proximal position with respect to the ablation arrangement. In this way transmurality of the ablation can be measured in order to monitor a progress of the ablation procedure, particularly if a circumferential ablation is achieved.

Alternatively or in addition, the expansion member includes a sensor device, for example a temperature sensor, for measuring a measurement quantity, for example temperature, in the vicinity of the ablation site. This allows for an additional monitoring of the ablation procedure.

Another measure to monitor progress and success of an ablation procedure is to monitor the sympathetic tone via the heart rate variability (HRV). For this, during an ablation procedure a frequency and amplitude analysis of an ECG signal is carried out. An increase in the heart rate variability indicates a reduction of the sympathetic tone, and vice versa.

In another aspect, a system for performing a cardiac sympathetic denervation intervention is provided, the system including a first catheter device of the type described previously for introduction into the aorta to perform a cardiac sympathetic denervation intervention at an ablation site within the aorta, and a second catheter device having a second ablation device for introduction into the pulmonary artery to perform a cardiac sympathetic denervation intervention at an ablation site within the pulmonary artery. Hence, within the system two catheter devices are used, one catheter device serving to perform a sympathetic denervation in the aorta, and another catheter device serving to perform a sympathetic denervation in the pulmonary artery. The second catheter device for the sympathetic denervation in the pulmonary artery may be functionally similar to the catheter device used in the aorta and may in particular include an ablation device having an expansion member similar as described above. By using two catheter devices at two different ablation sites, an effective ablation and hence interruption of the sympathetic nerves at the cardiac plexus may be achieved, which partially is located between the ascending aorta and the pulmonary trunk.

The ablation in the aorta and the ablation in the pulmonary artery may be performed synchronously by using the catheter devices at the same time, allowing for example a more efficient procedure and also to control the ablation procedures in dependence of each other.

Alternatively, one ablation procedure may be carried out before the other ablation procedure, such that for example first an ablation within the pulmonary artery and subsequently an ablation in the aorta may be carried out, or vice versa.

In yet another aspect, a method for performing a cardiac sympathetic denervation intervention using a catheter device is provided, the method including: introducing the catheter device in a retrograde direction into the aorta to perform a cardiac sympathetic denervation intervention at an ablation site within the aorta; expanding an expansion member of an ablation device of the catheter device from a compressed delivery state to an expanded ablation state to abut, in the expanded ablation state, an aortic wall section at the ablation site, the expansion member including an inner lumen providing for a blood flow passage through the ablation device in the expanded ablation state of the expansion member in an antegrade flow direction opposite to the retrograde direction; and applying an ablation action at the ablation site using an ablation arrangement arranged on the expansion member.

The various embodiments, advantages and aspects described above for the catheter device equally apply also to the system and the method.

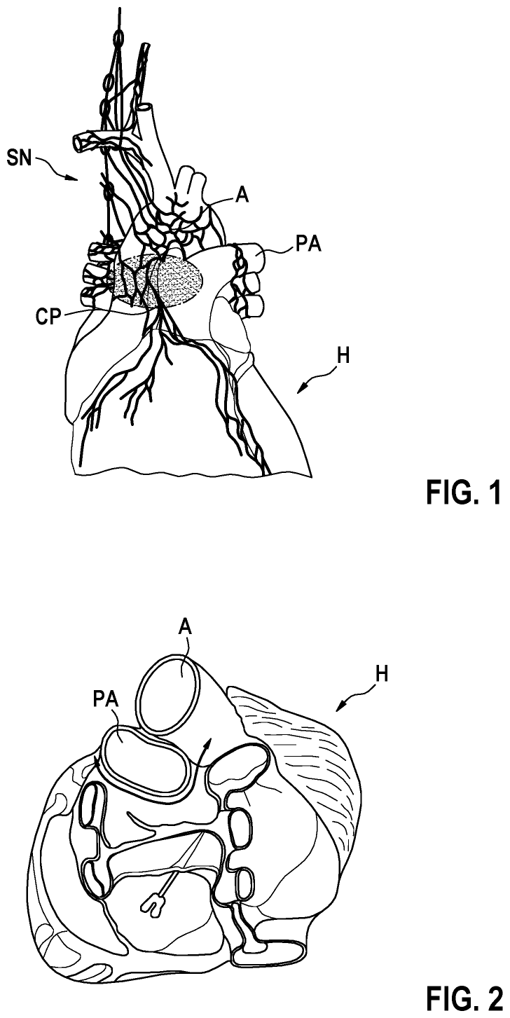

Referring now to FIG. 1, sympathetic nerves SN arise from the spinal cord, namely from the lateral horn of the thoracic spinal cord, and extend to the ganglium stellatum and the cervical ganglia. Sympathetic neurons herein form three cardiac nerves (superior, middle, inferior) which extend to the cardiac plexus CP and from there run alongside the great arteries to the base of the heart.

The sympathetic cardiac innervation contributes to cardiovascular and other diseases, such as heart failure, coronary artery disease, arrhythmias, stress-induced diseases, sleeping disorders, hypertension, kidney insufficiency, diabetes mellitus, metabolic syndrome depression, and strokes. Within a cardiac sympathetic denervation procedure, the sympathetic nerves (SN) innervating the heart are intersected typically at the sympathetic trunc on the left side or bilaterally, which nowadays is commonly accomplished by a video-assisted thoracoscopic surgical procedure which dissects the stellate ganglion removing the lower half along with a few ganglia below it.

In order to avoid risks involved with a surgical procedure, it is envisioned to provide for a cardiac sympathetic denervation using a minimally invasive catheter procedure.

Referring now to FIGS. 2 to 4, within the instant text it is proposed to use a catheter device 1 which is introduced for example transfemorally or radially in a retrograde direction R into the aorta to provide for an ablation using an ablation device 2 distal to the aortic valve AV as well as the left coronary artery LCA and the right coronary artery RCA, as this is illustrated in FIG. 4. Using the ablation device 2, an ablation within the aorta A is achieved such that the sympathetic nerves extending along the aorta A may be intersected in the region of the cardiac plexus CP to provide for a cardiac sympathetic denervation. The ablation target for the sympathetic denervation is the space between the ascending aorta and pulmonary trunk as indicated by the two arrows in FIG. 2.

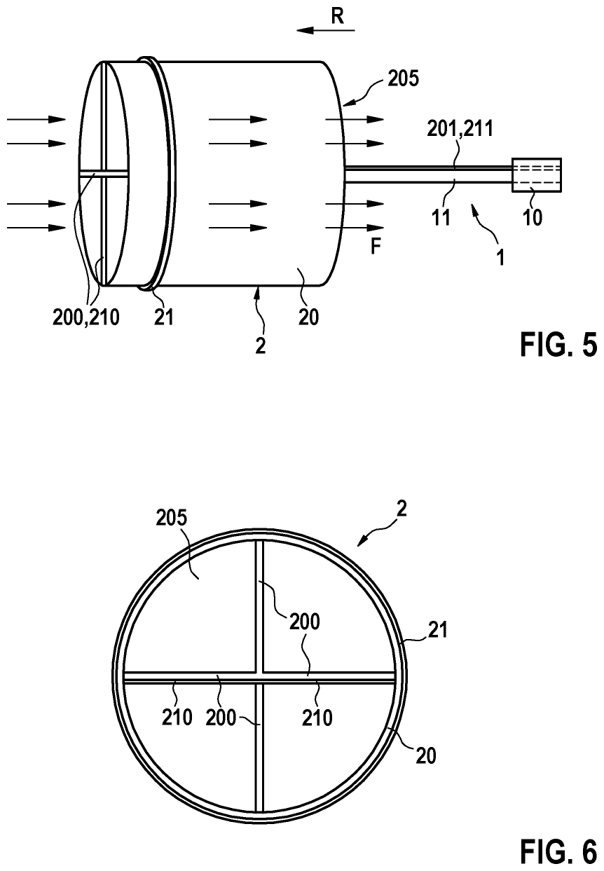

As illustrated in FIG. 4 and as schematically visualized in an embodiment in FIGS. 5 and 6, the catheter device 1 at its distal end includes an ablation device 2 having an expansion member 20 fixed to an inner catheter shaft 11 of the catheter device 1. The inner catheter shaft 11 is received within an outer catheter shaft 10, the inner catheter shaft 11 being axially movable along the axial direction of extension within the outer catheter shaft 10. In some embodiments the outer catheter may be optional.

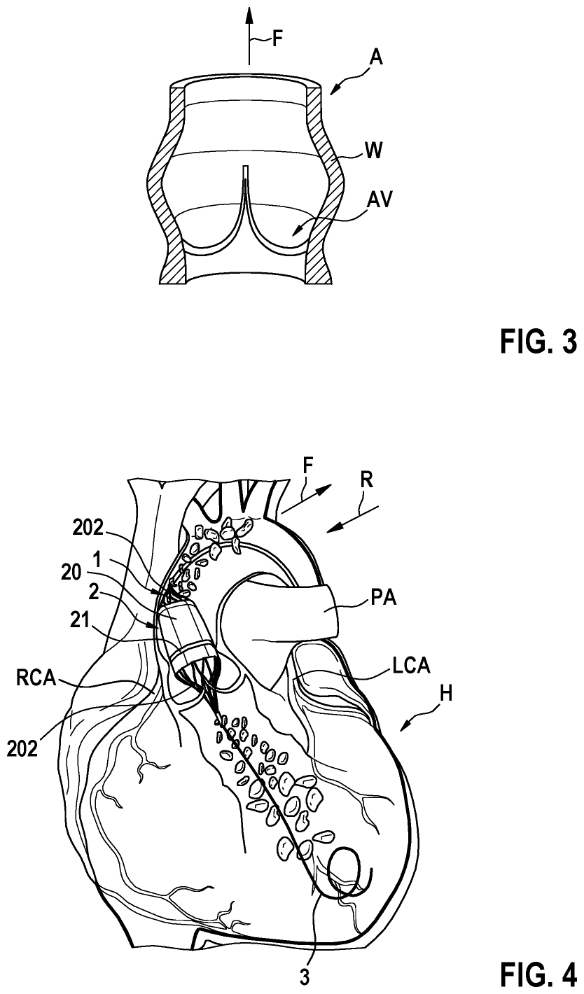

The expansion member 20 is radially expandable from a compressed delivery state to an expanded ablation state, as shown in FIGS. 5 and 6. In the compressed delivery state the expansion member 20 is received within the outer catheter shaft 10, specifically a receptacle formed on the outer catheter shaft 10, such that the catheter device 1 with the ablation device 2 arranged thereon may be delivered towards the ablation site. Once the ablation site is reached, the expansion member 20 may be caused to expand such that the expansion member assumes its expanded ablation state, in which the expansion member 20 abuts the aortic wall W of the aorta A in the vicinity of the aortic valve AV, as apparent from FIGS. 3 and 4.

The expansion member 20 carries an ablation arrangement 21 which serves to perform an ablation action on the aortic wall W when the expansion member 20 is in its expanded ablation state. The ablation arrangement 21 is arranged on an outer circumferential face of the expansion member 20 and, in the embodiment of FIGS. 5 and 6, circumferentially extends about the expansion member 20 such that the ablation arrangement 21 in a ring-like fashion abuts the aortic wall W along a circumferential ablation line.

In the embodiment of FIGS. 5 and 6, the expansion member 20 is formed by an inflatable balloon which has a generally cylindrical shape and confines, in its expanded state, an inner lumen 205, such that the expansion member 20 in its expanded state assumes the shape of a hollow tubing. Herein, fluid lines 200 extend in between the expansion member 20 and the inner catheter shaft 11 and are connected to a fluid line 201 extending along the catheter shaft 11, such that via the fluid lines 200, 201 a fluid may be delivered towards the expansion member 20 for inflating the expansion member 20 from the compressed delivery state to the expanded ablation state.

In the embodiment of FIGS. 5 and 6, multiple (in the shown embodiment four) fluid lines 200 extend radially to form a cross, as visible from the front-side view of FIG. 6, to provide for a fluid connection in between the fluid line 201 formed on the catheter shaft 11 and the expansion member 20. The fluid lines 200 may also serve for structural stabilization between the catheter shaft 11 and the expansion member 20. In addition further stabilizing elements (not shown) may be used to connect the catheter shaft 11 and the expansion member 20.

In the embodiment of FIGS. 5 and 6, the ablation arrangement 21 may be configured to provide for e.g. a cryoablation. For this, the ablation arrangement 21 may have the shape of a fluid line which circumferentially extends about the expansion member 20 and which is connected to fluid lines 210 which connect the ablation arrangement 21 to fluid lines 211 arranged on the inner catheter shaft 11. Via the fluid lines 210, 211 a cooling agent may be delivered towards and passed through the ablation arrangement 21, for example liquid carbon dioxide at a temperature of −56° C., to cool tissue in the vicinity of the ablation arrangement 21 and to hence provide for a cryoablation.

As the expansion member 20 is hollow on its inside, blood may flow through the ablation device 2 via a blood flow passage being formed within an inner lumen 205 along an antegrade flow direction F in the expanded ablation state of the expansion member 20, such that the aortic blood flow is not blocked while performing an ablation procedure.

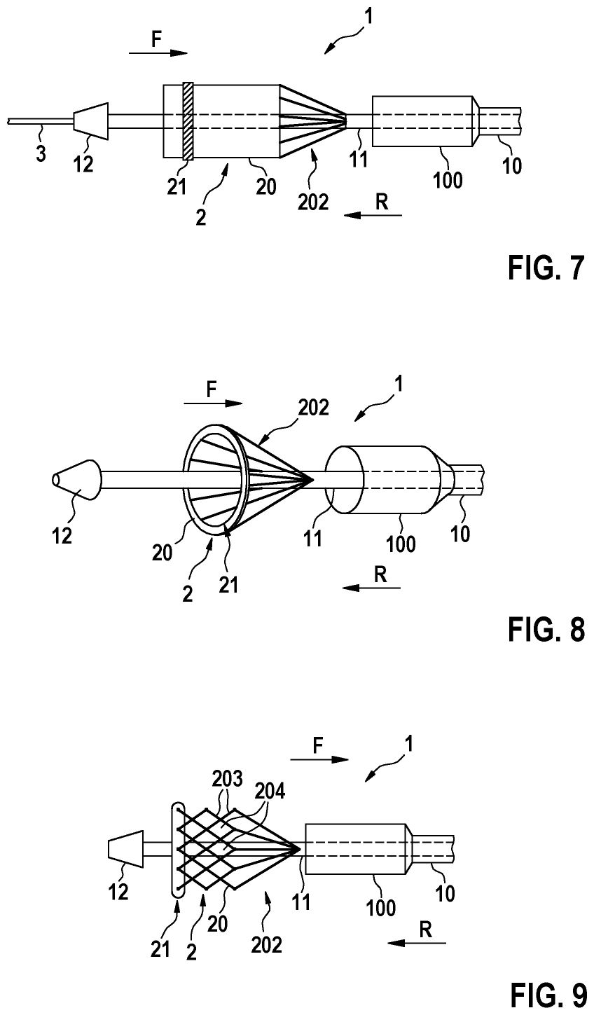

Referring now to FIG. 7, in one embodiment the expansion member 20, as for example shown in FIGS. 5 and 6, is connected to the catheter shaft 11 by connection members 202 in the shape of a multiplicity of flexible connection lines, struts or tines, which hold the expansion member 20 in place with respect to the catheter shaft 11.

As this is visible from FIG. 4 in combination with FIG. 7, such connection members 202 may be arranged on one axial end of the expansion member 20, as illustrated in FIG. 7, or on both axial ends of the expansion member 20, as shown in FIG. 4, to provide for a connection of the expansion member 20 to the catheter shaft 11 in the expanded ablation state of the ablation device 2.

In the embodiment of FIG. 7, a catheter tip 12 is arranged on the catheter shaft 11, the catheter shaft 11 extending through the expansion member 20 such that the catheter tip 12 is placed to distally of the expansion member 20.

The catheter shaft 11 may for example form an inner lumen through which a guide wire 3 extends, the catheter shaft 11 being slidable on the guide wire 3 such that the catheter device 1 may be guided towards the ablation site in the aorta A along the guide wire 3 for delivering the ablation device 2 towards the ablation site for performing an ablation procedure.

In the embodiment of FIG. 7, a receptacle 100 in the shape of a substantially cylindrical housing is formed on a distal end of the outer catheter shaft 10, the ablation device 2 being received within the receptacle 100 in a compressed delivery state of the expansion member 20. By axially moving the catheter shaft 11 with respect to the catheter shaft 10 the ablation device 2 may be delivered from the receptacle 100 such that the expansion member 20 may be expanded by inflating the balloon-shaped expansion member 20 to assume the expanded ablation state. In addition, after completing the ablation the expansion member 20 may be deflated and once more received in the receptacle 100 to remove the ablation device 2 from the patient.

An ablation arrangement 21 is arranged on an outer circumferential face of the expansion member 20, as illustrated in FIG. 7, such that the ablation arrangement 21 abuts the aortic wall W at the ablation site in the aorta A in the expanded ablation state of the expansion member 20.

Whereas in the embodiments of FIG. 7 and FIGS. 5 and 6 the expansion member 20 includes a substantially cylindrical shape having an axial extension exceeding the diameter of the expansion member 20 in the expanded ablation state, in another embodiment shown in FIG. 8 the expansion member 20 has the shape of a torus which extends in a ring-like manner around the catheter shaft 11 and which is fixed to the catheter shaft 11 by connection members 202 in the shape of connection lines, struts or tines.

An ablation arrangement 21 is arranged on the outer circumference of the expansion member 20 to abut the aortic wall W in the expanded ablation state of the expansion member 20.

Again, a receptacle 100 in the shape of a substantially cylindrical housing is formed on a distal end of the catheter shaft 10, the expansion member 20 being received within the receptacle 100 in a compressed delivery state for delivering the ablation device 2 towards an ablation site in a retrograde direction R, and for removing the ablation device 2 from the patient after completing the ablation.

In another embodiment shown in FIG. 9 the ablation device 2 includes an expansion member being formed by a stent-like structure like a frame cut from a tube or a wireframe made from wires. The wireframe consists of struts 203 connected to each other to form nodes, such that the wireframe forms closed cells 204, which in a multiplicity of rows are aligned to extend about the inner catheter shaft 11. Connection members 202, which may be integrally formed with the wireframe of the expansion member 20, serve to connect the expansion member 20 to the catheter shaft 11, such that the expansion member 20 in the shape of the wireframe is connected to and held on the catheter shaft 11. In an embodiment of the expansion member 20, the frame or wireframe of the expansion member 20 is attached with its proximal end and with its distal end to the catheter shaft 11, whereas the connection to the catheter shaft 11 is such, that the frame or wireframe can slide along the catheter shaft 11 with either its proximal end or its distal end. This slidable connection can be for example realized using a ring like structure surrounding the catheter shaft 11 and being connected to the frame or wireframe. By moving the ring like structure along the shaft 11, the frame or wireframe expands mechanically. In a further embodiment of the expansion member 20, the proximal end of the frame or the wireframe of the expansion member 20 can be connected to the inner catheter shaft 11 and the distal end of the frame or the wireframe can be attached to a further catheter, slidable arranged inside of the inner catheter shaft 11. This allows the expansion member 20 to be expanded by moving the additional inner catheter relative to the inner catheter shaft 11.

Whereas in the embodiments of FIGS. 5 to 8 the expansion member 20 is expanded by inflating the expansion member 20 using an inflation fluid, such as a gas or a liquid, in the embodiment of FIG. 9 the ablation device 2 is self-expendable in that the expansion member 20, once withdrawn from the receptacle 100, automatically assumes the expanded ablation state due to its self-elasticity. For this, the expansion member 20 may for example be integrally formed from an elastic metal material, in particular a nitinol material.

As the axial length of the expansion member 20 in the shape of the wireframe may be shortened when deploying the expansion member 20 to assume its expanded state, the catheter device 1 may include a so-called foreshortening compensation mechanism, as it for example is described in U.S. Pat. No. 10,172,732 B2.

It is conceivable to support expansion of the expansion member 20 in the shape of the stent-like wireframe by additional expansion means, such as for example an additional inflatable balloon or the like.

An ablation arrangement 21 is arranged on the expansion member 20 of FIG. 9. The ablation arrangement 21 may be formed by a fluid channel through which a cooling agent may be guided for providing for a cryoablation. Alternatively, the ablation arrangement 21 may be formed by a multiplicity of individual elements, such as Peltier elements for providing for a cryoablation, or electrodes for providing for a radiofrequency ablation, which are connected to the struts 203 of the expansion member 20, for example at nodes at which the struts 203 are interconnected.

Generally, the ablation arrangement 21 in all of the embodiments described above may be formed as a fluid line extending circumferentially about the expansion member 20 for guiding a cooling agent such as liquid carbon dioxide for providing for a cryoablation along an ablation line defined by the ablation arrangement 21. Fluid lines, as illustrated by the lines 210, 211 in FIGS. 5 and 6, herein are connected to the fluid line of the ablation arrangement 21 to form a circular flow, such that the cooling agent may be passed through the fluid line of the ablation arrangement 21 via the catheter shaft 11.

Alternatively, the ablation arrangement 21 may be configured to provide for a cryoablation using one or multiple Peltier elements.

Yet alternatively, the ablation arrangement 21 may be configured to provide for a thermal ablation using radiofrequency signals, such as an injection of an alternating RF current, or microwave signals for locally heating and hence ablating tissue contacted by the ablation arrangement 21.

Yet alternatively, the ablation arrangement 21 may be configured to provide for an ultrasound ablation, in particular a high-intensity focused ultrasound (HIFU) ablation.

Yet alternatively, the ablation arrangement 21 may be configured to provide for a chemical ablation or an electroporation.

For providing, for example, a chemical ablation, the ablation arrangement 21 may include one or multiple delivery devices for delivering a chemical agent to tissue at the ablation site. The ablation arrangement 21 for this may include for example a multiplicity of delivery openings, for example in the shape of micro-needles, by which the chemical agent may be injected into the tissue, wherein in this case the ablation arrangement 21 is formed by a fluid line through which the chemical agent may be guided and injected into the tissue via the delivery devices.

For the chemical ablation, the ablation arrangement 21 may form a multiplicity of micro-channels, micro-pumps and micro-valves as used in microfluidics technology. Within microfluidics technology fluids are constrained to a small, typically a sub-millimeter scale at which capillary penetration governs the transport of the fluid, which may be employed to deliver a chemical agent using the ablation arrangement 21 in a precisely controlled fashion.

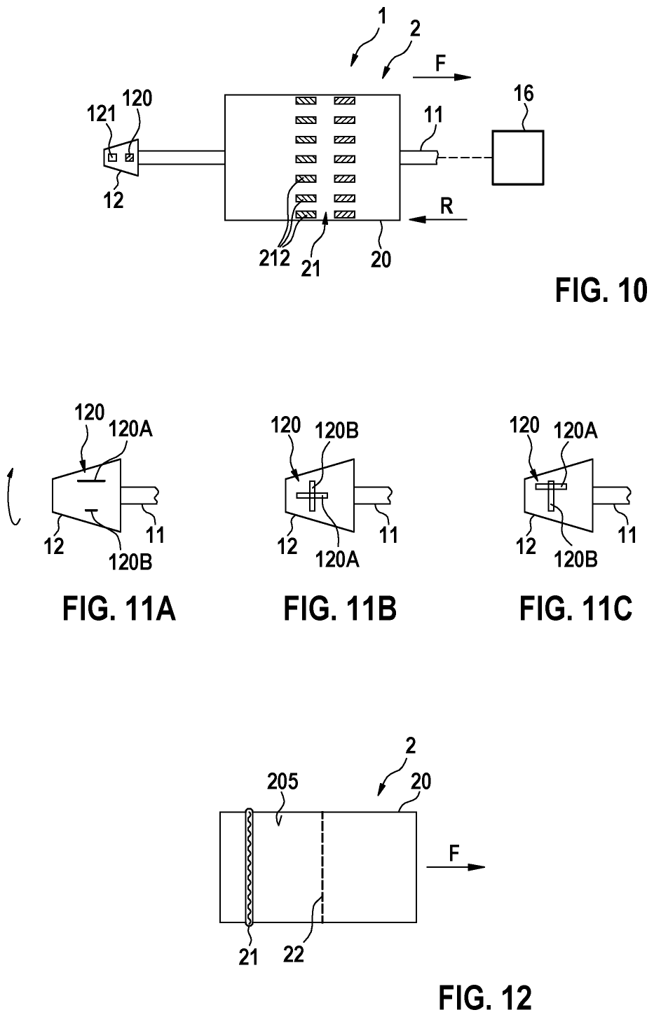

Referring now to FIG. 10, the ablation arrangement 21 may generally consist of a single element extending for example circumferentially about the expansion member 20, or by an arrangement of individual ablation members 212 spatially distributed on the outer circumferential face of the expansion member 20, as shown in FIG. 10. The ablation members 212 may for example have the shape of Peltier elements, electrodes or chemical delivery devices and may for example be arranged on the outer circumferential face of the expansion member 20 to form an arrangement of rows and columns, such that the ablation members 212 are spatially distributed in a regular or irregular fashion on the expansion member 20.

The ablation members 212 may be commonly controlled for providing for an ablation action using an electronic control device 16 connected to the catheter device 1. The control device 16 herein may for example control the ablation members 212 to perform an identical ablation action at all ablation members 212, or may individually control the ablation members 212 to provide for a locally distinguished ablation in which regions may individually be subjected to ablation by a certain group of the ablation members 212 of the ablation arrangement 21.

If the ablation members 212 shall be individually controlled using the control device 16 for spatially controlling ablation at the ablation site and confining the ablation to certain regions, the rotational orientation of the catheter device 1 must be known. For this, the catheter device 1 is beneficially inserted into the patient such that it assumes a defined rotational orientation at the ablation site. For monitoring the rotational orientation, herein, a rotational orientation indicator 120 may be arranged for example at the catheter tip 12, the rotational orientation indicator 120 being shaped such that the rotational orientation of the catheter device 1 at its distal end may be assessed using a imaging technology, such as an X-ray technology.

Referring now to FIGS. 11A to 11C, the rotational orientation indicator 120 may for example be formed by indicator sections 120A, 120B embedded in the catheter tip 12 or formed on an outer face of the catheter tip 12, the indicator sections 120A, 120B being formed for example from a radiopaque material such that they are visible for example under X-ray. The indicator sections 120A, 120B herein are arranged on the catheter tip 12 such that their combined image within e.g. X-ray imaging varies with the rotational orientation of the catheter tip 12, the indicator sections 120A, 120B together forming for example a cross as shown in FIG. 11B if the catheter tip 12 and hence the catheter device 1 is at its correct, desired rotational orientation. If the catheter device 1 however is oriented at an incorrect rotational angle, as illustrated in FIG. 11A (showing a rotation with respect to the rotational orientation of FIG. 11B by roughly 90°) and FIG. 11C, the image of the indicator sections 120A, 120B substantially differs from a cross, such that a user immediately and intuitively is guided to rotate the catheter device 1 until the correct rotational orientation of FIG. 11B is reached. Alternatively the rotational orientation indicator may be arranged at the expansion member 20. In this case the rotational orientation indicator may for example be formed by indicator sections embedded in the expansion member 20 or formed on an outer face of the expansion member 20.

Referring now again to FIG. 10, in addition a tracking device 121 such as a marker or a tracking sensor may be placed for example on the catheter tip 12, the tracking device 121 serving to allow for a tracking to facilitate the delivery of the catheter device 1 and to monitor the position of the catheter device 1 within the patient during delivery using a tracking technology. Alternatively the tracking device can also be located on the expansion member 20. The marker for tracking can be made for example from a radiopaque material such that it is visible for example under X-ray. The tracking sensor for example can be a magnet sensor to localize the expansion member 20 or the tip of the catheter device in an external magnetic field.

By controlling the ablation members 212 of FIG. 10 using the control device 16, an automatic control of the ablation becomes possible, for example by controlling the ablation members 212 individually and independently of each other to provide for an ablation action. This may take place in a closed-loop operation, in which the ablation procedure is monitored and is controlled in dependence of the progress of ablation. Further, using for example a mapping device, the control may automatically take place such that an ablation is performed at the site of the sympathetic nerves in the vicinity of the cardiac plexus, the ablation hence being confined to a locally defined area by individually controlling the ablation members 212.

Referring now to FIG. 12, in all of the embodiments described above a filter element 22 may be arranged within the expansion member 20, the filter element 22 serving to filter particles from a blood flow flowing through the expansion member 20 in the expanded ablation state. In the expanded ablation state the expansion member 20 abuts the aortic wall W at the ablation site, a blood flow passage being formed within an inner lumen 205 of the expansion member 20 such that blood may flow in an antegrade flow direction F through the expansion member 20. In order to avoid a transport of particles through the aorta, for example emboli, the filter element 22 spans across the inner lumen 205 of the expansion member 20, such that particles may effectively be filtered out from the blood flow within the expansion member 20.

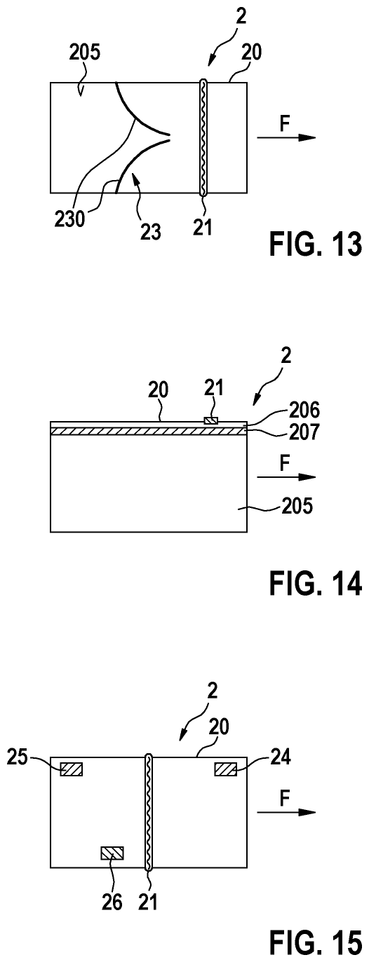

Referring now to FIG. 13, in all of the embodiments described above a valve arrangement 23 may be placed within the expansion member 20, the valve arrangement 23 being formed by one or multiple leaflets 230 providing for an artificial aortic valve within the inner lumen 205 of the expansion member 20. In particular if the ablation device 2 shall be placed within the native aortic valve AV for providing an ablation action in the aorta A in the vicinity of the aortic valve AV, the valve arrangement 23 may facilitate that during the ablation regular cardiac operation may be maintained, the valve arrangement 23 temporarily serving as aortic valve during the ablation procedure.

Referring now to FIG. 14, showing a schematic cross-sectional view through an embodiment of an ablation device 2, the expansion member 20 may include an isolation layer 207 for example placed at the inside of an outer wall 206 of the expansion member 20, the isolation layer 207 providing for an isolation in between the ablation arrangement 21 at the outside of the expansion member 20 and the inner lumen 205 and hence a blood flow flowing through the inner lumen 205 during an ablation procedure. If the ablation arrangement 21 for example provides for a cryoablation, the isolation layer 207 may be formed to provide for a thermal isolation. If the ablation arrangement 21 is configured to provide for a radiofrequency ablation, the isolation layer 207 may provide for a thermal isolation and in addition an electrical isolation.

Referring now to FIG. 15, in all of the embodiments described above electrodes 24, 25 may be placed on the expansion member 20, wherein a first electrode 24 may be placed proximally of the ablation arrangement 21 and hence at an antegrade position with respect to the ablation line defined by the ablation arrangement 21, and a second electrode 25 may be placed distally of the ablation arrangement 21 and hence at a retrograde position with respect to the ablation line defined by the ablation arrangement 21. Using the electrodes 24, on the expansion member 20, for example impedance measurements may be conducted during an ablation procedure, such that in this way a progress and success of an ablation procedure can be monitored.

Alternatively or in addition, progress and success of an ablation procedure can be assessed by analyzing the heart rate variability (HRV) for example by conducting a frequency analysis of an ECG signal before and after an ablation procedure. By the heart rate variability the sympathetic tone can be estimated, an increase in the heart rate variability indicating a reduction of the sympathetic tone and vice versa.

Referring again to FIG. 15, alternatively or in addition a sensor device 26, such as for example a temperature sensor, may be placed on the expansion member 20, the sensor device 26 serving to measure a measurement quantity, such as temperature, during an ablation procedure to monitor progress and success of the ablation procedure.

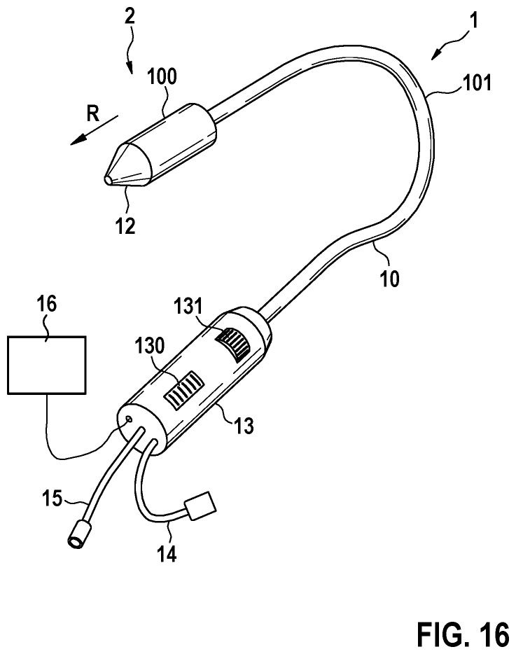

Referring now to FIG. 16, a catheter device 1 implementing an ablation device 2 as described for any of the embodiments referred to above may include a handle 13 allowing to control the catheter device 1 during insertion into a patient and during an ablation procedure. In the embodiment of FIG. 16, the handle 13 is placed at a proximal end of the catheter device 1, the ablation device 2 being placed at a distal end of the catheter device 1 and being received, in its compressed delivery state, in a receptacle 100 formed on the outer catheter shaft 10. The catheter device 1 with the ablation device 2 arranged thereon can be inserted into a patient in a retrograde direction R for delivering the ablation device 2 towards an ablation site in the aorta of the patient.

In the embodiment of FIG. 16, the outer catheter shaft 10 may for example include a steerable section 101, which may be deflected in order to guide and steer the catheter device 1 through vessels of the patient for delivering the ablation device 2 towards the ablation site.

A deflection of the steerable section 101 may for example be controlled by an actuation element 131 in the shape of a turning knob or the like arranged on the handle 13.

An additional actuation element 130 arranged on the handle 13 may serve to control an axial position of the inner catheter shaft 11 with respect to the outer catheter shaft 10, in particular for delivering the ablation device 2 from the receptacle 100 at the ablation site.

Connectors 14, 15 may be arranged at the handle 13 for example to inject an inflation fluid towards the expansion member 20 for inflating the expansion member 20, or to inject a cooling agent into the ablation arrangement 21 for providing for a cryoablation. In addition a guide wire lumen may run along the catheter to direct the catheter over the wire into the desired target region of the body.

A control device 16 is connected to the handle 13 to control the ablation operation of the catheter device 1.

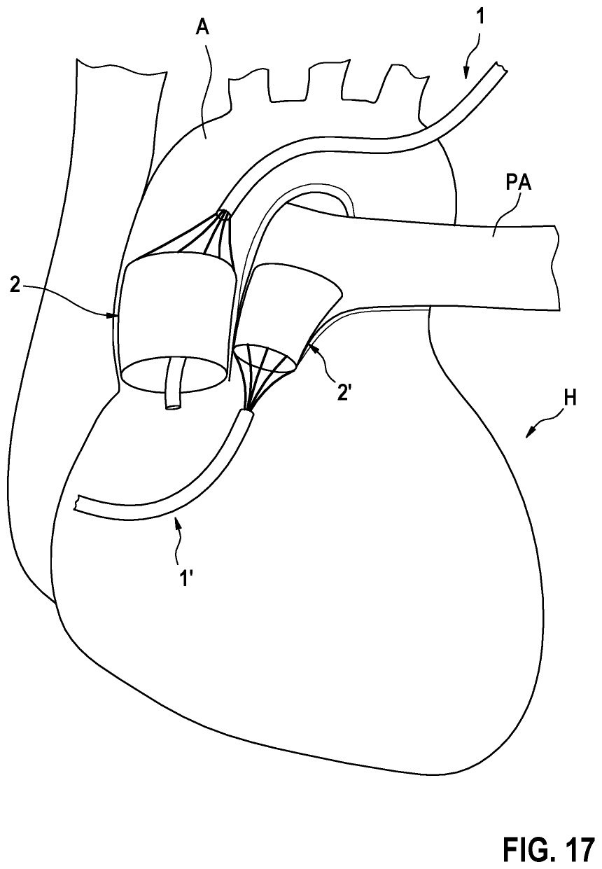

Referring now to FIG. 17, a catheter device 1 as described above serves to provide for an ablation within the aorta A by inserting the ablation device 2 towards an ablation site in the vicinity of the aortic valve. An ablation herein may be performed by the catheter device 1 alone, or within an ablation system in combination with another, second catheter device 1′, as illustrated in FIG. 17.

The other, second catheter device 1′ may include an ablation device 2′ which is functionally similar to the ablation device 2 as described above, but which may be configured for insertion—for example via the vena femoralis, the vena jugularis or the vena subclavia—into the pulmonary artery PA for providing an ablation within the pulmonary artery PA.

Within an ablation system the catheter devices 1, 1′ may be used synchronously to provide a synchronous ablation in the aorta A and in the pulmonary artery PA. It however is also conceivable to first apply an ablation in the pulmonary artery PA and subsequently in the aorta A, or vice versa, such that ablation at different locations takes place in multiple steps.

The idea underlying the invention is not limited to the embodiments described above, but may be implemented in another fashion.

A sympathetic denervation by ablation of sympathetic nerve fibers using a catheter device may be used for therapy and/or prevention of multiple diseases, such as peripheral arterial diseases, sleeping disorder, sleep apnea, hyperhidrosis, kidney insufficiency, depression, posttraumatic stress disorder (PTSD), diabetes mellitus, metabolic syndrome, strokes, and acute heart failure.

By achieving a sympathetic denervation using a catheter-based ablation a secure and beneficially controllable denervation procedure may be provided, in particular in comparison to surgical cardiac sympathetic denervation procedures.

While specific embodiments of the present invention have been shown and described, it should be understood that other modifications, substitutions and alternatives are apparent to one of ordinary skill in the art. Such modifications, substitutions and alternatives can be made without departing from the spirit and scope of the invention, which should be determined from the appended claims.

Various features of the invention are set forth in the appended claims

### LIST OF REFERENCE NUMERALS

1, 1′ Catheter device
10 Catheter shaft (outer catheter)
100 Receptacle
101 Steerable section
11 Catheter shaft (ablation catheter)
12 Catheter tip
120 Rotational orientation indicator
120A, B Indicator sections
121 Tracking device (marker or sensor)
13 Handle
130, 131 Actuation element
14, 15 Connector
16 Control device
2, 2′ Ablation device
20 Expansion member
200, 201 Fluid line
202 Connection members
203 Struts
204 Cells
205 Inner lumen
206 Wall section
207 Thermal isolation layer
21 Ablation arrangement
210, 211 Fluid line
212 Ablation member
22 Filter element
23 Valve arrangement
230 Leaflets
24, 25 Electrode
26 Sensor device
3 Guide wire
A Aorta
AV Aortic valve
CP Cardiac plexus
F Flow direction
H Heart
PA Pulmonary artery
R Retrograde direction
SN Sympathetic nerves
W Aortic wall section

1. A catheter device for performing a cardiac sympathetic denervation intervention, the device comprising: an ablation device, the ablation device comprising an expansion member that is expandable from a compressed delivery state to an expanded ablation state and an inner lumen defining a blood flow passage through the ablation device in the expanded ablation state in an antegrade flow direction (F), wherein the catheter device is configured for introduction into a vessel and the expansion member is configured to abut, in the expanded ablation state, a wall section (W), and wherein the expansion member carries an ablation arrangement.

2. The catheter device according to claim 1, wherein the catheter device comprises a first catheter shaft and a second catheter shaft received within the first catheter shaft and being axially movable within the first catheter shaft, wherein the ablation device is fixed to the second catheter shaft such that the ablation device is axially movable with respect to the first catheter shaft.

3. The catheter device according to claim 2, wherein the first catheter shaft comprises a receptacle configured to receive the expansion member in the compressed delivery state, wherein the expansion member, by axially moving the second catheter shaft relative to the first catheter shaft, is deliverable from the receptacle to transfer the expansion member from the compressed delivery state to the expanded ablation state.

4. The catheter device according to claim 1, wherein the expansion member is self-expandable.

5. The catheter device according to claim 1, wherein the expansion member is inflatable using a fluid to expand from the compressed delivery state to the expanded ablation state.

6. The catheter device according to claim 1, wherein the expansion member comprises a shape of an inflatable balloon defining said inner volume within.

7. The catheter device according to claim 1, wherein the expansion member comprises a wire frame shape.

8. The catheter device according to claim 1, wherein the ablation arrangement is arranged on an outer face of the expansion member.

9. The catheter device according to claim 1, wherein the ablation arrangement extends circumferentially about the antegrade flow direction on the expansion member.

10. The catheter device according to claim 1, wherein the ablation arrangement is configured to perform cryoablation, radiofrequency ablation, microwave ablation, ultrasound ablation, and/or chemical ablation.

11. The catheter device according to claim 1, wherein the ablation arrangement comprises a multiplicity of ablation members distributed on the expansion member.

12. The catheter device according to claim 11, comprising a control device configured to independently control individual ones of the multiplicity of ablation members.

13. The catheter device according to claim 1, wherein the expansion member comprises at least one of a filter element configured to filter a blood flow passing through the inner lumen to remove particles from the blood flow, a valve arrangement comprising at least one leaflet configured to provide an artificial valve for a blood flow passing through the inner lumen, an electrode arrangement configured to conduct impedance measurements, and a sensor device configured to sense a measurement quantity.

14. A system for performing a cardiac sympathetic denervation intervention, wherein the vessel is an aorta (A), the system comprising a first catheter device according to claim 1 configured to be introduced into the aorta (A) to perform a cardiac sympathetic denervation intervention at an ablation site within the aorta (A), and a second catheter device having a second ablation device configured to be introduced into the pulmonary artery (PA) to perform a cardiac sympathetic denervation intervention at an ablation site within the pulmonary artery (PA).

15. A method for performing a cardiac sympathetic denervation intervention using a catheter device, comprising: introducing the catheter device in a retrograde direction (R) into a vessel to an ablation site to perform a cardiac sympathetic denervation intervention at the ablation site; expanding an expansion member of an ablation device of the catheter device from a compressed delivery state to an expanded ablation state to abut, in the expanded ablation state, a wall section (W) at the ablation site, the expansion member comprising an inner lumen defining a blood flow passage through the ablation device in the expanded ablation state of the expansion member in an antegrade flow direction (F) opposite to the retrograde direction (R); and applying an ablation action at the ablation site using an ablation arrangement arranged on the expansion member.
